(19) United States
(12) Patent Application Publication
(10) Pub. No.: US 2004/0167547 A1
(43) Pub. Date: Aug. 26, 2004

(54) Surgical suture placement device

(76) Inventors: Richard M. Beane, Hingham, MA (US); James Alan Crunkleton, Weston, MA (US); Douglas M. Dahl, Weston, MA (US); James L' Esperance, Shrewsbury, MA (US)

Correspondence Address: FISH & RICHARDSON PC, 225 FRANKLIN ST, BOSTON, MA, 02110, US

(21) Appl. No.: 10/613,502

(22) Filed: Jul. 3, 2003

Related U.S. Application Data
(60) Provisional application No. 60/394,131, filed on Jul. 3, 2002.

(51) Int. Cl. A61B 17/04

(52) U.S. Cl. 606/144

(57) ABSTRACT

The invention provides a surgical suture placement device that includes a handle assembly rotatably secured to an elongated hollow outer tube extending from the handle assembly. Also included is a suturing assembly rotatably secured to the distal end of the elongated hollow outer tube and a hollow flexible tube connecting the handle assembly to the suture assembly by traversing the inside of the elongated hollow outer tube. The device can be used for placing a suture through the urethra and bladder neck to perform an urethrovesical anastomosis, e.g., after removal of the prostate.

### CROSS-REFERENCE TO RELATED APPLICATION

This application claims priority from U.S. Provisional Patent Application Serial No. 60/394,131, filed Jul. 3, 2002, the subject matter of which is incorporated herein by reference in its entirety.

### TECHNICAL FIELD

This invention relates to devices and methods for performing anastomoses.

### BACKGROUND

Over 100,000 radical prostatectomies are performed each year. The vast majority of these procedures are performed using an open surgical technique, despite proven advantages of the laparoscopic radical prostatectomy including improved visualization, substantially reduced blood loss, and reduced patient morbidity. The most technically challenging step of the laparoscopic technique is the urethrovesical anastomosis. The technical difficulty of this anastomosis serves as a major barrier to broader-scale use of laparoscopy to perform the radical prostatectomy.

### SUMMARY

The invention includes devices and methods for placing a suture to connect two tissues, e.g., two tubular tissues. For example, the device can be used to connect the urethra to the bladder neck to perform an urethrovesical anastomosis, e.g., after removal of the prostate. The anastomosis consists of reattachment of the urethra to a larger diameter opening in the bladder neck. The invention can also be used to perform anastomoses in other vessels and lumens and for difficult open prostatectomies.

In one aspect, the invention features a surgical suture placement device that includes a handle assembly, which can optionally rotate; an elongated hollow outer tube connected at a proximal end to the handle assembly; a suturing assembly rotatably secured to a distal end of the elongated hollow outer tube; and a hollow inner tube, e.g., a flexible tube, located within the hollow outer tube and connecting the handle assembly to the suture assembly by traversing the inside of the elongated hollow outer tube.

In this device, the suturing assembly can include a holding member for removably holding a needle. The device can also include the needle itself, wherein the needle is hollow and has an open, sharp-tipped distal end, e.g., with rounded edges that avoid damage to the suture material that passes through the needle. The hollow needle can be configured to enable a suture to pass through a portion of the needle, the suture extending from an aperture on a surface at a proximal end of the needle to an opening at a sharpened distal end of the needle.

The surgical suture placement device can further include a thin flexible rod arranged within the hollow inner tube, wherein the rod is connected at a proximal end to the handle assembly and has a distal end configured for connection to a needle located within the suturing assembly. The surgical suture placement device can also include a needle plunger arranged within the handle, wherein a distal end of the needle plunger attaches to a proximal end of a needle by means of a thin rod, wherein the thin rod traverses the interior of the flexible hollow tube.

The surgical suture placement device can also include a spring that biases the needle plunger into an extended position and the needle into a retracted position. In addition, the suturing assembly can be angled away from a longitudinal axis of the elongated hollow outer tube, e.g., a longitudinal axis of the suture assembly can be at an angle of 45° to a longitudinal axis of the elongated hollow outer tube.

In certain embodiments in which the handle rotates, rotating the handle concurrently rotates the hollow inner tube and the suture assembly as a unit.

The suture assembly of the new device can include a needle cover attached to the elongated hollow outer tube; a needle guide with a suture aperture attached to the needle cover; and a suture holder attached to the needle guide, wherein the needle guide is secured between the needle cover and the suture holder.

In another aspect, the invention features methods of suturing a first tissue to a second tissue by obtaining a new suture placement device as described herein and loading the device with a needle and suture. Then one moves a distal portion of the suture placement device into proximity of tissue to be sutured; positions the suturing assembly at a proper angle; extends a suture needle through a portion of a first tissue; retracts the needle leaving a first loop of suture extending from the first tissue; extends the needle through a portion of a second tissue; retracts the needle leaving a second loop of suture extending from the second tissue; and connects the first and second loop of suture to suture the first tissue to the second tissue.

In these methods, the first and second loops of suture can be connected with a knot or with a crimping device. The method can be a surgical anastomosis, and the anastomosis can be performed during a tubal ligation, heart bypass surgery, coronary artery bypass graft, or urethrovesical anastomosis. In these methods, the suture placement device need not be removed from the proximity of the tissue until suturing is complete. The first and second tissues can be different (e.g., the urethra and the bladder neck), or they can be different sections of the same tissue. In certain methods, the tissue to be sutured can be adjacent to a body cavity (e.g., the bladder), the suture placement device is inserted into the body cavity, and then is not removed from the body cavity until the suturing is complete.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic cross-section of a suture placement device.

FIGS. 2a and 2b are schematic diagrams of a protracted and extended needle, respectively, in a rotating suture assembly of a suture placement device.

FIGS. 3a to 3c are schematic diagrams of a protracting needle used in a new suture placement device.

FIG. 4 is a schematic diagram of a hollow needle.

FIG. 5 is a disassembled view of a rotating suture assembly.

FIGS. 6a and 6b are schematic diagrams of how a suture knot is formed using the new suture placement device.

### DETAILED DESCRIPTION

The invention greatly reduces the technical expertise required for performing laparoscopic radical prostatectomies. The invention enables more surgeons to take advantage of the proven advantages of laparoscopic techniques. Typically, six to ten stitches are used for an anastomosis. Each stitch requires placing suture material through the urethra and bladder neck, approximating the two structures, and securing the ends of the suture, typically by tying a knot. Thus, placement of each stitch can be considered in two steps: (1) placement of suture material through a first tissue and a second tissue, and (2) securing the ends of the suture, such as with a knot. The invention disclosed herein assists in the first step, placement of suture material though the urethra and bladder neck. This step currently requires very sophisticated laparoscopic skills and is not feasible for most urologists. Products are presently available to assist in the second step. These devices can use a crimp to secure the ends of the suture. Such devices are described, e.g., in U.S. Pat. Nos. 5,931,844, 6,086,608 and 6,200,329.

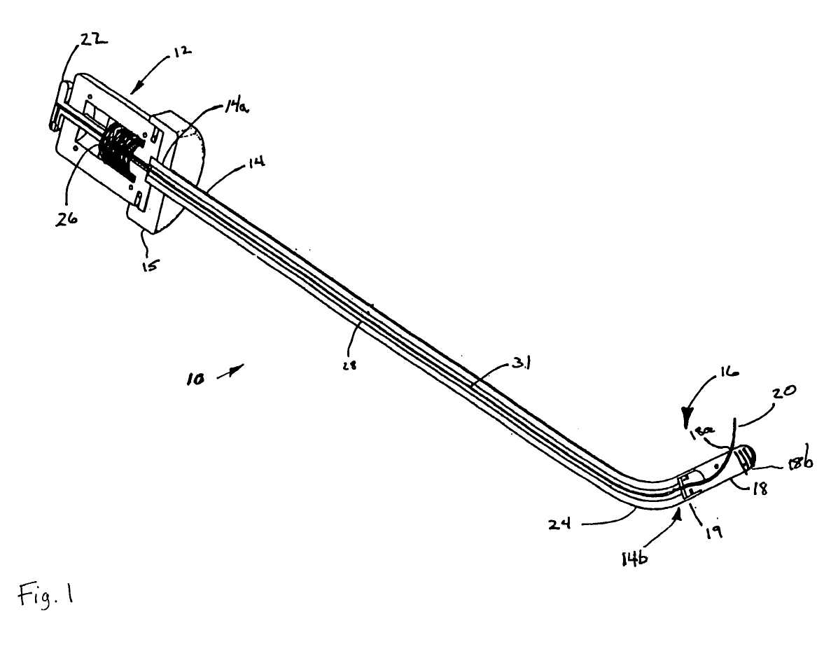

FIG. 1 shows a surgical suture placement apparatus, designated generally by 10, for assisting in an anastomosis, e.g., an urethrovesical anastomosis. The device has the general shape of a urethral sound that is a device commonly used by urologists. The device includes an elongated hollow outer tube 14, which has a predetermined diameter, e.g., about a 24 French (0.301 in) diameter, a handle assembly 12, and a rotating suturing assembly 16. The device also includes a needle plunger 22 and a needle 20 (shown partially extended). The needle plunger is spring loaded 26 so that the needle 20 is normally in the fully retracted position.

The surgical suture placement device 10 has a handle assembly 12 rotatably connected to the proximal end of the elongated hollow outer tube 14. The elongated hollow outer tube 14 is rigidly attached to a sleeve 15 at the proximal end 14a. The handle assembly 12 rotates within the sleeve 15 to provide rotation to the rotating suturing assembly. Sleeve 15 is cylindrical in shape with a diameter greater than the diameter of the elongated hollow outer tube 14.

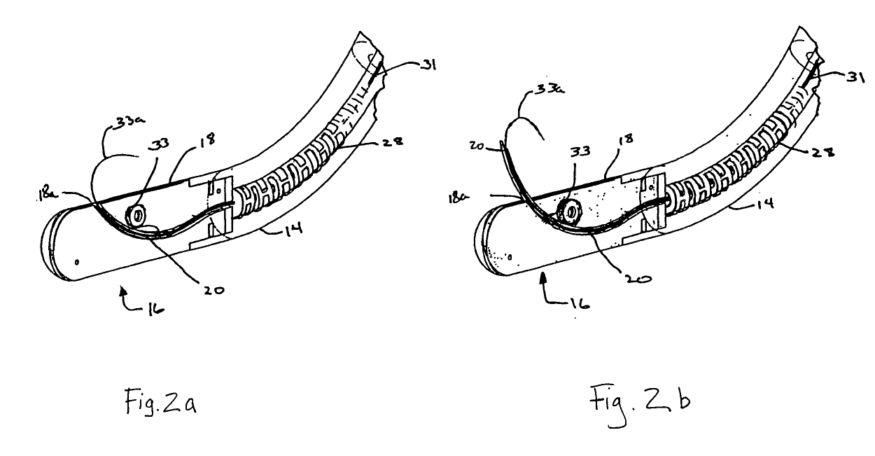

The rotating suturing assembly 16 is rotatably attached to the distal end 14b of the elongated hollow outer tube 14. A hollow inner tube 28 having an outer diameter smaller than the inner diameter of the elongated hollow outer tube 14, traverses the elongated hollow outer tube 14 internally to make a second connection between the handle assembly 12 and the suturing assembly 16. The second connection between the handle assembly 12 and the suturing assembly 16 is a nonrotatable connection. Thus, turning the handle 12 turns the suturing assembly 16 in the same direction. As shown in FIGS. 2a and 2b, hollow inner tube 28 can include ridges to allow the tube to flex yet maintain rotational rigidity.

The connections discussed above can be made by a variety of methods, e.g., fitted sleeves, clamping, mated male and female threads, soldering, gluing, or a gear assembly.

The suturing assembly 16 includes a holding member 18 which is rotatably connected to the elongated hollow outer tube 14. The holding member 18 (also shown in section) holds a suturing needle 20. The holding member 18 includes a narrow opening 18a which traverses one side of the holding member 18 and allows for protrusion of the needle 20 from the holding member 18 during suturing. The needle 20 protrudes from the holding member 18 at a set angle to the longitudinal axis of the holding member. The angle can be, for e.g., 30′-75′.

In another variation of the invention, the holding member 18 includes markings 18b that are used to position the holding member 18 at the proper angle and insertion depth in the subject. The markings 18b are etched or otherwise placed during manufacturing on to the holding member 18 and are viewed through a laparoscopic opening in the subject's abdomen. During suture placement through the urethra, most of the rotating suturing assembly 16 is inside the urethra and the location of the opening 18a is not visible; thus, markings 18b are needed to indicate the angle that the needle 20 will be deployed and to provide an approximate location of the needle 20 to the urethra opening.

The needle 20 is connected to the spring-loaded plunger 22, which extends through the handle assembly 12, by e.g., a thin rod 31, which connects to the distal end of the plunger 22 and the proximal end of the needle 20. Pressure exerted downward on the plunger 22 forces it into the handle assembly, causing a force to be exerted on the thin rod 31, which in turn exerts a force on the needle 20, resulting in the needle 20 protracting from the holding member 18 through the narrow opening 18a. In the absence of a force, the spring 26 is selected and arranged to maintain the plunger 22 in an extended position and the needle 20 in a retracted position within the holding member 18.

The handle assembly 12 can rotate in a clockwise or counter-clockwise direction. In one variation of the invention, rotation of the handle assembly 12 concurrently rotates the connected hollow inner tube 28, while the elongated hollow outer tube 14 remains stationary. This movement also rotates the holding member 18, which is securely connected to the hollow inner tube 28, in the same direction.

The distal end 14b of the elongated hollow outer tube 14 can be angled away from the central axis of the outer tube 14. The angle 24 occurs prior to the junction 19 of the elongated hollow outer tube 14 with the holding member 18. The bend of the elongated hollow outer tube 14 is selected for the specific surgery, e.g., a 45° angle from the longitudinal axis of the elongated hollow outer tube 14. The elongated hollow outer tube 14 is made of a rigid material to maintain the angle 24, and also to allow the device 10 to be placed into a subject without distorting the structure of the device 10. The outer tube 14 can be manufactured of, e.g., stainless steel or other metals, and plastic polymers.

When the elongated hollow outer tube 14 is angled, the hollow inner tube 28 and the thin rod 31 must be bendable at their distal ends. During assembly, the hollow inner tube 28 is inserted into the elongated hollow outer tube 14, and the thin rod 31 is inserted into the hollow inner tube 28, resulting in both components assuming the shape of the elongated hollow outer tube 14. The hollow inner tube 28 must be sufficiently flexible (e.g., it can be made of plastic, a spring, fiberglass, biopolymers, or rubber) to assume the angle of the elongated hollow outer tube 14, but have sufficient rotational rigidity to rotate the holding member 18. The thin rod 31 must also have the flexibility (and thus can be manufactured of, e.g., plastic, a spring, fiberglass, a biopolymer, or rubber) to assume the angle 24 of the elongated hollow outer tube 14, but have the longitudinal rigidity to transfer a protraction force from the spring-loaded plunger 22 to the needle 20.

A variation of the invention is that the distal end of the elongated hollow outer tube 14 is not angled from its longitudinal axis, but remains straight.

FIGS. 2a and 2b are schematic cross-sectional views of the suture assembly 16 connected to a portion of an elongated hollow outer tube 14, a hollow inner tube 28, and a rod 31. FIG. 2a shows the needle 20 in a recessed position (with only suture material 33a extending out of the device). The suture assembly 16 contains a spool 33 of suture material 33a that is threaded through the interior of the hollow needle 20. Applying pressure to the spring loaded 26 plunger 22 forces the rod 31 to extend the connected needle 20 through the narrow opening 18a in the holding member 18, and out of the device as diagramed in FIG. 2b.

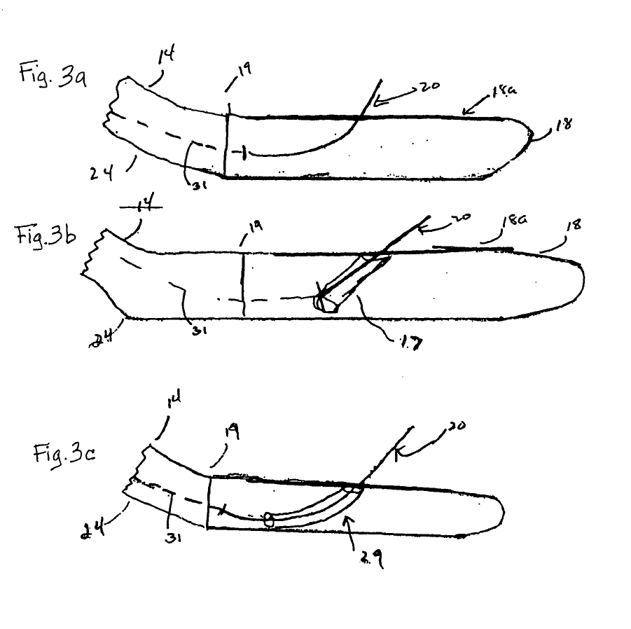

Referring to FIG. 3a, the needle 20 can be extended from the holding member 18 by a variety of methods. In the variation shown in FIG. 3a, the needle 20 has a curved, banana shape, and is made of a rigid material, e.g., stainless steel or other metals, a plastic, or a polymer. The thin rod 31 extends through the angled elongated hollow outer tube 14 and pushes the needle 20 outward and at an angle from the holding member 18. The hollow inner tube 28 is not shown in this drawing.

FIG. 3b shows another embodiment in which the needle 20 is straight and is made of a rigid material. The thin rod 31 pushes the needle 20 through a straight hollow guide 17, to protract out of and at an angle to the holding member 18. The hollow inner tube 28 is not shown in this drawing.

FIG. 3c shows another embodiment in which the needle 20 is made of a material rigid enough to penetrate tissue, but flexible enough to adapt to a curved, hollow guide 29. The thin rod 31 pushes the needle 20 through the curved guide 29 and out of the narrow opening 18a at an angle to the holding member 18. Again, the hollow outer tube 28 is not shown in this drawing.

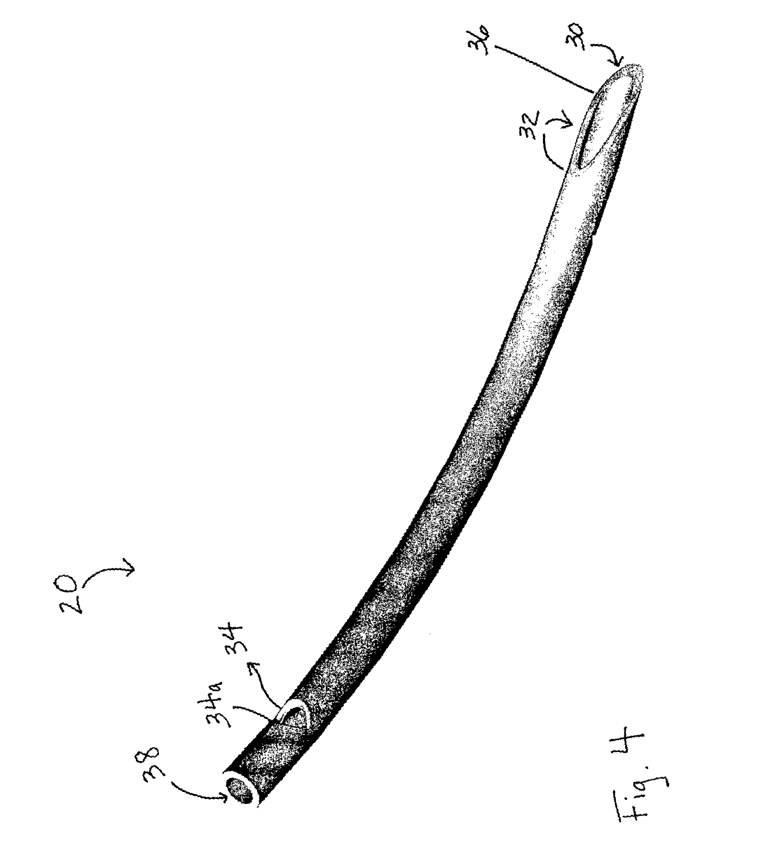

FIG. 4 shows needle 20 of the rotating suturing assembly 16. The needle is hollow with open proximal 38 and distal 36 ends. In use, the proximal end 38 is connected to the distal end of the thin rod 31. The surface near the proximal end 38 of the needle 20 has an aperture 34, with rounded edges 34a, traversing the outer to the inner surface. This aperture 34 is where the suture material 33a enters the needle 20; the rounded edges 34a protect the suture material from damage. The distal end 36 of the needle 20 contains the sharp, pointed end 30 of the needle, which allows the needle 20 to smoothly traverse a tissue. The edges 32 are also rounded, preventing damage to the suture. The suturing material extends through the hollow needle 20 and exits through the distal end 36. In this variation of the invention, the needle 20 is slightly curved.

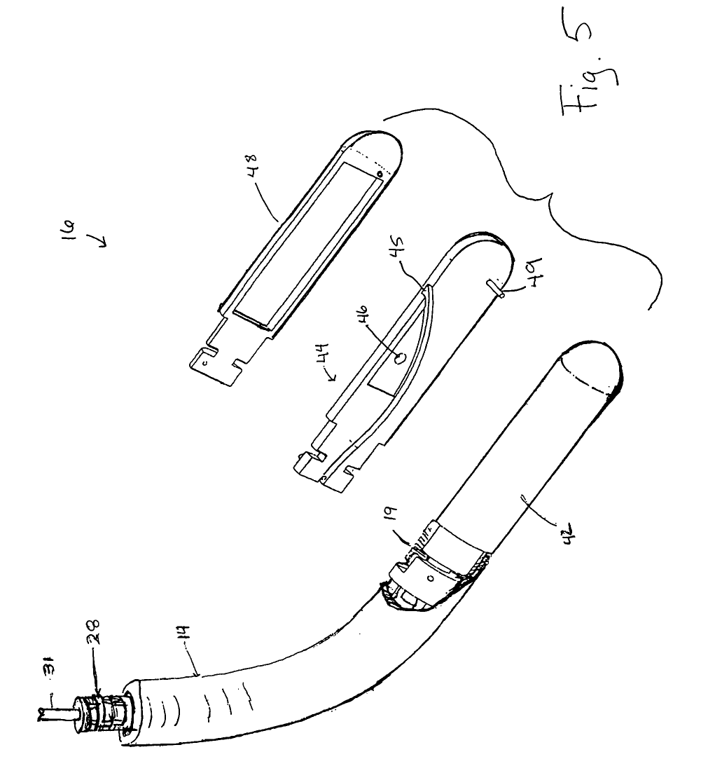

FIG. 5 is a disassembled view of the rotating suturing assembly 16. The bottom part shown in the figure is the needle cover 42 with an attached elongated hollow inner tube 28 which can rotate inside the hollow tube 14 to provide rotation to rotating suturing assembly 16. The middle part shown in the figure is the needle guide 44, which contains and guides the needle 20 through the slotted groove 45. The needle guide also has a suture hole 46 for the suture material to extend from the suture holder 48 (top part shown in the figure) to the hollow needle 20. Thirty inches or more of suture material can be loaded in a spool 33 or in figure-eight configuration in the suture holder 48 for deployment through the needle guide hole 46 into the needle 20. The suture material then extends through the hollow needle 20 to the sharp-edge end 30 of the needle 20 where a short length of suture material is deployed. Pin 49 holds the various parts together.

All components of the suture placement device can be manufactured by methods known in the art. All components of the device can be made reusable, therefore, each component can be sterilized without damage to the component, e.g., UV irradiation, autoclaving, exposure to alcohol. Alternatively, all components of the device can be made disposable and replaceable.

Methods of Use

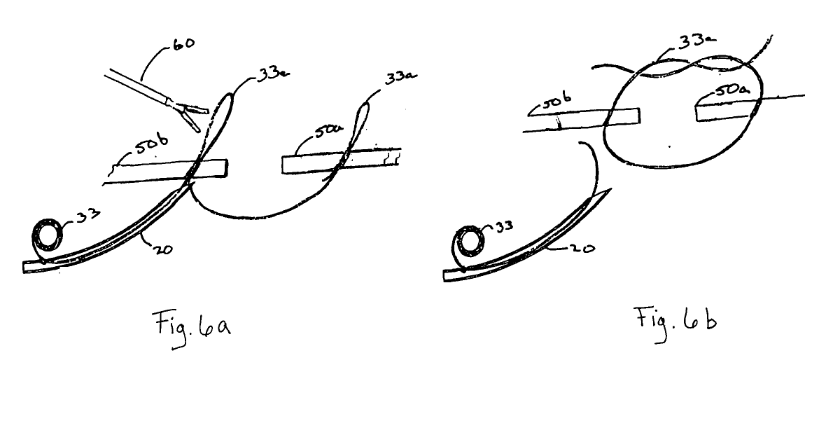

FIGS. 6a and 6b show the general use of the suturing placement device 10 to place a suture during an anastomosis. FIG. 6a shows the placement of the suture before crimping. The tissues to be connected, 50a and 50b, have been pierced by the needle 20 leaving a loop of suturing material 33a, which is fed from a spool 33 of suturing material, extending through the tissue wall. A laparoscopic instrument 60 makes a single cut through the looped suturing material 33a severing it from the needle. In FIG. 6b, each loop of the severed suturing material 33a that extends through each tissue wall 50a and 50b has been entertwined by a laparoscopic instrument 60 (not shown in FIG. 6b) to form a knot. Alternatively, the cut ends can be crimped using standard techniques and devices.

One specific use of the suture placement device 10 is for placing suturing material through a urethra and a bladder neck after radical prostatectomy. The device 10 can be inserted into the penis and extended to the site of anastomosis through the transected proximal urethra after the prostate has been removed. Placing the device 10 in the urethra provides stability to the device 10 and places the device 10 in close proximity to the site of anastomosis. Inserting the device 10 through the penis also allows unhindered use of the laparoscopic instruments during the anastomotic procedure to manipulate tissue or suture material as necessary. Markers 18b on the suturing assembly 16 are viewed with a laparoscope and are used to position the suturing assembly 16 in the urethra at the proper angle and insertion depth. Once the appropriate position is achieved, the needle plunger 22 is pressed to extend the needle 20 with suturing material to push through the wall of the urethra.

The needle 20 is then retracted by reducing force on the spring-loaded needle plunger 22, leaving a length of suturing material extending from the outside of the urethra. This length of suturing material is then pulled using a laparoscopic instrument to obtain an appropriate length of suturing material for the stitch. The device 10 is then positioned inside the bladder neck. Again, the needle 20 is extended through the bladder wall and retracted, leaving a length of suturing material extending from the outside of the bladder neck. The suture can be then cut (when an interrupted anastomosis is desired), the ends of the suture are pulled to approximate the urethra and bladder neck, and the suturing material ends are secured using a knot or a crimping device. The rotating suturing assembly 16 is then positioned at a different location and the process is repeated to apply the second suture. The surgeon can repeat the process to apply as many sutures as desired, and the sutures are applied without removing the device from the penis. Alternatively, a “running” anastomosis can be performed where the suture is not cut until the stitching is completed. As another alternative, a “tennis racquet” anastomosis, which is well known to those skilled in the art, can be performed by attaching the urethra to a short circumference of bladder neck and then suturing bladder neck to bladder neck. Other anastomosis alternatives can be achieved using the new device as the surgeon desires.

The suture placement device 10 can be used in many other locations within the body, essentially in any situation where a tube needs to be connected to a larger tube or a larger lumen (e.g., blood vessel to blood vessel, blood vessel to organ, bowel to bowel). A similar device can be used to connect a tube to a larger tube outside the body as well. In addition, the new device can be used to deliver a cytotoxin or radiolabel to a site (e.g., a tumor) by placing the compound into the rotating suture assembly and delivering the compound instead of, or at the same time as, delivering the suture.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A surgical suture placement device comprising:
a handle assembly;
an elongated hollow outer tube connected at a proximal end to the handle assembly;
a suturing assembly rotatably secured to a distal end of the elongated hollow outer tube; and
a hollow inner tube located within the hollow outer tube and connecting the handle assembly to the suture assembly by traversing the inside of the elongated hollow outer tube.

2. The surgical suture placement device of claim 1, wherein the suturing assembly comprises a holding member for removably holding a needle.

3. The surgical suture placement device of claim 2, further comprising the needle, and wherein the needle is hollow and has an open, sharp-tipped distal end.

4. The surgical suture placement device of claim 3, wherein the opened, sharp-tipped distal end of the needle comprises rounded edges.

5. The surgical suture placement device of claim 1, wherein the suturing assembly further comprises a hollow needle configured to enable a suture to pass through a portion of the needle, the suture extending from an aperture on a surface at a proximal end of the needle to an opening at a sharpened distal end of the needle.

6. The surgical suture placement device of claim 1, further comprising a thin flexible rod arranged within the hollow inner tube wherein the rod is connected at a proximal end to the handle assembly and has a distal end configured for connection to a needle located within the suturing assembly.

7. The surgical suture placement device of claim 1, wherein the hollow inner tube is flexible.

8. The surgical suture placement device of claim 1, further comprising a needle plunger arranged within the handle, wherein a distal end of the needle plunger attaches to a proximal end of a needle by means of a thin rod, wherein the thin rod traverses the interior of the flexible hollow tube.

9. The surgical suture placement device of claim 8, further comprising a spring that biases the needle plunger into an extended position and the needle into a retracted position.

10. The surgical suture placement device of claim 1, wherein the suturing assembly is angled away from a longitudinal axis of the elongated hollow outer tube.

11. The surgical suture placement device of claim 1, wherein a longitudinal axis of the suture assembly is at a 45° angle to a longitudinal axis of the elongated hollow outer tube.

12. The surgical suture placement device of claim 1, wherein the handle is rotatable.

13. The surgical suture placement device of claim 1, wherein rotating the handle concurrently rotates the hollow inner tube and the suture assembly as a unit.

14. The surgical suture placement device of claim 1, wherein the suture assembly comprises:
a needle cover attached to the elongated hollow outer tube;
a needle guide with a suture aperture attached to the needle cover; and
a suture holder attached to the needle guide, wherein the needle guide is secured between the needle cover and the suture holder.

15. A method of suturing a first tissue to a second tissue, the method comprising;
obtaining a suture placement device of claim 1 and loading the device with a needle and suture;
moving a distal portion of the suture placement device into proximity of tissue to be sutured;
positioning the suturing assembly at a proper angle;
extending a suture needle through a portion of a first tissue;
retracting the needle leaving a first loop of suture extending from the first tissue;
extending the needle through a portion of a second tissue;
retracting the needle leaving a second loop of suture extending from the second tissue; and
connecting the first and second loop of suture to suture the first tissue to the second tissue.

16. The method of claim 15, wherein the first and second loops of suture are connected with a crimping device.

17. The method of claim 15, wherein the method is a surgical anastomosis.

18. The method of claim 17, wherein the anastomosis is performed during a tubal ligation, heart bypass surgery, coronary artery bypass graft, or urethrovesical anastomosis.

19. The method of claim 17, wherein the anastomosis is a urethrovesical anastomosis.

20. The method of claim 15, wherein the suture placement device is not removed from the proximity of the tissue until suturing is complete.

21. The method of claim 15, wherein the first and second tissues are different.

22. The method of claim 15, wherein the first and second tissues are different sections of the same tissue.

23. The method of claim 15, wherein the tissue to be sutured is adjacent to a body cavity, the suture placement device is inserted into the body cavity, and is not removed from the body cavity until the suturing is complete.

24. The method of claim 23, wherein the body cavity is the bladder, the first tissue to be sutured is a urethra, and the second tissue to be sutured is a bladder neck.
